(19) United States
(12) Patent Application Publication
(10) Pub. No.: US 2011/0045106 A1
(43) Pub. Date: Feb. 24, 2011

(54) COFFEE EXTRACT

(75) Inventors: Rachid Bel-Rhlid, Savigny (CH); Karin Kraehenbuehl, Fully (CH); Christophe Cavin, Montreux (CH); Thomas Wolfgang Raab, Grandvaux (CH); Nicolas Page, Lausanne (CH)

Correspondence Address: K&L Gates LLP, P.O. Box 1135, CHICAGO, IL, 60690, US

(73) Assignee: NESTEC S.A., Vevey (CH)

(21) Appl. No.: 12/990,593

(22) PCT Filed: Mar. 12, 2009
(86) PCT No.: PCT/EP09/52936

§ 371 Date: Nov. 1, 2010

(30) Foreign Application Priority Data
Apr. 30, 2008 (EP) 08155448.7

(51) Int. Cl. A61K 36/74; A61P 17/00; A61P 3/10; A61P 25/00; A61P 29/00; A61P 3/04; A61P 35/00; A61P 19/08; A61P 39/06; A23F 5/02; A23F 5/26

(52) U.S. Cl. 424/725; 426/45

(57) ABSTRACT

The present invention relates to a method of producing a coffee extract with antioxidant and anti-inflammatory properties and uses of the extract of the invention. The coffee extract comprises caffeic acid and/or ferulic acid and can be produced by hydrolysing chlorogenic acids present in coffee extract, e.g. with a microorganism or an enzyme. The coffee extract of the invention may e.g. be used as an ingredient of a food or beverage product.

### FIELD OF THE INVENTION

The invention relates to a method of producing an improved coffee extract with antioxidant and anti-inflammatory properties, methods of producing the extract, and uses of the extract of the invention.

### BACKGROUND OF THE INVENTION

Coffee and coffee active compounds such as caffeine and diterpenes (e.g. cafestol, kahweol) have been shown to induce detoxifying enzymes (e.g. glutathione-S-transferases GST) in rodents and in humans (Cavin C. et al, 1998. The coffee-specific diterpenes cafestol and kahweol protect against aflatoxin Bl-induced genotoxicity trough a dual mechanism. Carcinogenesis 19, 1369-1375; Cavin, C. et al, 2003. Coffee diterpenes prevent benzo[a]pyrene genotoxicity in rat and human culture systems. Biochemical Biophysical Research Communication 306, 488-495; Huber, W. et al. 2002a. Enhancement of the chemoprotective enzymes glucuronyl transferase and glutathione transferase in specific organs of the rat by the coffee components kahweol and cafestol. Archive of Toxicology 76, 209-217). Increased GST activity by coffee has been further demonstrated in human following consumption of 800 ml of coffee for 5 days (Steinkellner, H. et al. 2005. Coffee consumption induces GSTP in plasma and protects lymphocytes against (+/−)-anti-benzo [a] pyrene-7,8-dihydrodiol-9,10-epoxide induced DNA-damage: results of controlled human intervention trials. Mut. Res. 591 264-275). In addition, Richelle et al., J. Agric. Food Chem. 49 3438-42, (2001) showed the lag time of LDL oxidation in vitro is significantly increased by coffee under standard cup serving. An increased total antioxidant capacity was also observed in human plasma in vivo after 200 ml of coffee consumption (Natella et al., J. Agric. Food Chem. 50, 6211-16, 2002).

This kind of antioxidant activity is known to protect against “oxidative stress” by reducing damaging free radicals that may be implicated e.g. in cancer, heart disease, degenerative brain disorders and ageing.

To increase the health benefits of food and beverage products there is a desire to produce products with an increased antioxidant activity, as well as other beneficial biological activities, and to find natural sources of antioxidants and other compounds with beneficial biological activities, that can be used to enhance the properties of food and beverage products as well as e.g. cosmetic and medical products. The antioxidant properties can be intrinsic to the nature of the molecule itself or can be mediated by the induction of the natural defenses against oxidative stresses.

### SUMMARY OF THE INVENTION

The inventors have now found a method of producing a coffee extract wherein the amount of caffeic acid is at least 1 milligram per gram of dry matter and/or the amount of ferulic acid is at least 0.5 milligram per gram of dry matter; and that such a coffee extract has improved antioxidant and anti-inflammatory properties as compared to conventional coffee extracts. Accordingly, the invention relates to a method of producing a coffee extract, comprising the following steps: a) extracting coffee beans with water to produce a coffee extract; and b) treating the coffee extract to hydrolyse chlorogenic acids to generate phenolic acids. In further embodiments the invention relates to uses of the coffee extract of the invention; a method of producing a food or beverage product; and the resulting food or beverage product.

### BRIEF DESCRIPTION OF THE FIGURES

FIG. 1: Western Blot gels showing protein expression of GST subunits (GSTA4, GSTP1) and Heme-Oxygenase-1 (HO-1) in rat primary heptocytes treated with 200 and 400 ug/ml NESCAFE RED CUP® (extract of roasted coffee beans) not treated to hydrolyse chlorogenic acids, and 200 and 400 ug/ml NESCAFE PROTECT® treated with Lactobacillus johnsonii, as well as control samples not treated with coffee extract. For details see example 1.

FIG. 2: Western Blot showing induction of detoxifying enzyme expression (GSTP1; NQO1) in the liver of male rats fed in their diet for 2 weeks with 5% of NESCAFE RED CUP® (extract of roasted coffee beans) not treated to hydrolyse chlorogenic acids (RN), NESCAFE PROTECT® (a co-extract of green and roasted coffee beans) not treated to hydrolyse chlorogenic acids (P); and NESCAFE PROTECT® treated with Lactobacillus johnsonii (La1-P). For details see example 1.

### DETAILED DESCRIPTION OF THE INVENTION

The present invention is related to a coffee extract with improved antioxidant and anti-inflammatory properties.

In one embodiment of the invention the coffee extract comprises at least 1 milligram of caffeic acid per gram of dry matter, such as at least 2, at least 5, at least 10, or at least 25 milligram of caffeic acid per gram of dry matter. In another embodiment the coffee extract comprises at least 0.5 milligram of ferulic acid per gram of dry matter, such as at least 1, at least 2, at least 5 or at least 10 milligram of ferulic acid per gram of dry matter. In a further embodiment the ratio of the amount of caffeoyl quinic acids and diesters to the amount of caffeic acid is less than 100 (weight/weight), such as less than 50, less than 10, or less than 1. In a still further embodiment the ratio of the amount of feruloyl quinic acids and diesters to the amount of ferulic acid is less than 30 (weight/weight), such as less than 10, less than 5 or less than 1.

The coffee extract of the invention may be an extract of green coffee beans and/or roasted coffee beans. Numerous methods for producing coffee extracts are known in the art.

The invention further relates to a method of producing a coffee extract, comprising the following steps: a) extracting coffee beans with water to produce a coffee extract; and b) treating the coffee extract to hydrolyse chlorogenic acids to phenolic acids.

The coffee beans to be extracted may be whole or ground. In one embodiment of the invention green coffee beans are co-extracted with roasted coffee beans, i.e. green and roasted coffee beans are extracted simultaneously in the same extraction system to yield a mixed extract. The most volatile aroma components may be stripped from the beans before extraction, e.g. if the extract is to be used for the production of pure soluble coffee. Methods for stripping of volatile aroma components are well known in the art, e.g. from EP 1078576.

Extraction of coffee beans with water and/or steam is well known in the art, e.g. from EP 0916267. The extract may undergo a concentration step and may be dried before the treatment to hydrolyse chlorogenic acids, e.g. by spray drying or freeze drying. If the extract has been dried it may be resuspended if required to effect the treatment to hydrolyse chlorogenic acids.

Chlorogenic acids are a family of esters formed between trans-cinnamic acids and quinic acid. Chlorogenic acids are naturally present in coffee, mainly as mono- and di-esters of quinic acid and phenolic groups (e.g. caffeic, ferulic, coumaric, methoxycinnamic) attached to different positions. By the method of the invention chlorogenic acids (quinic acid esters) in the coffee extract may be hydrolysed to generate phenolic acids, e.g. the chlorogenic acids 3-, 4-, or 5-caffeoyl quinic acid and diesters, and 3-, 4-, or 5-feruloyl quinic acid and diesters may be hydrolysed to generate caffeic acid and ferulic acid, respectively. In a preferred embodiment of the method caffeoyl quinic acids and/or diesters are hydrolysed to generate caffeic acid, and/or feruloyl quinic acid and/or diesters are hydrolysed to generate ferulic acid. In one embodiment of the invention the treatment to hydrolyse chlorogenic acids to generate phenolic acids is performed so as to achieve an amount of caffeic acid in the treated extract of at least 1 milligram of caffeic acid per gram of dry matter, such as at least 2, at least 5 or at least 10 milligram of caffeic acid per gram of dry matter. In another embodiment of the invention the treatment to hydrolyse chlorogenic acids to generate phenolic acids is performed so as to achieve an amount of ferulic acid in the treated coffee extract of at least 0.5 milligram of ferulic acid per gram of dry matter, such as at least 1, at least 2 or at least 5 milligram of ferulic acid per gram of dry matter. In a further embodiment at least 20%, such as at least 30%, at least 50% or at least 75% of caffeoyl quinic acids and diesters, and/or feruloyl quinic acids and diesters present in the coffee extract is hydrolysed by the treatment to hydrolyse chlorogenic acids to generate phenolic acids.

The treatment of the extract to hydrolyse chlorogenic acids to phenolic acids may be performed after or during the extraction. The extract may be separated from the extracted coffee beans before, during or after the treatment to hydrolyse chlorogenic acids. In one embodiment the extract is kept separate from the extracted coffee beans after the treatment to hydrolyse chlorogenic acids, i.e. the extract is not brought into contact with the extracted coffee beans again after the treatment to hydrolyse chlorogenic acids. Separation of the extract from the extracted coffee beans may be performed by any suitable method, e.g. filtration or centrifugation. The separation may be performed to the extent practically and economically feasible and needed in view of the desired use of the extract. The separation may thus not be 100% complete, e.g. a minor part of undissolved material from the beans may still be present with the extract after separation.

The hydrolysis of chlorogenic acid may be performed by any suitable method. In one embodiment of the invention the hydrolysis is performed by incubating or fermenting the coffee extract with a microroganism capable of hydrolysing chlorogenic acid in the coffee extract. Microorganisms capable of hydrolysing chlorogenic acid may e.g. be identified as disclosed in the examples of this application. Suitable microorganisms may be selected from yeasts, fungi or bacteria. Suitable microorganisms may e.g. be an Aspergillus; such as e.g. Aspergillus oryzae, a Lactobacillus, such as e.g. L. johnsonii (CNCM I-1225); a Bifidobacterium, such as e.g. B. lactis (CNCM I-3446), or a yeast such as e.g. Saccharomyces cerevisiae. Incubation or fermentation may be performed by inoculating the coffee extract with a microorganism capable of hydrolysing chlorogenic acids under conditions suitable for the growth of the specific microroganism for the time necessary to achieve the required hydrolysis of chlorogenic acids. The specific conditions can easily be determined by the skilled person, e.g. with reference to the examples contained herein. In another embodiment of the invention the hydrolysis of chlorogenic acids is performed by the use of non-replicative microorganism, e.g. lysed cells. By incubating coffee extract with lysed cells under suitable conditions, enzymes present in the cell lysate may hydrolyse chlorogenic acids to generate phenolic acids. Suitable cells may e.g. be cells of the microroganisms mentioned above. Suitable methods for producing cell lysate are known in the art.

The amount of microorganism and conditions of the fermentation should be suitable to achieve the desired hydrolysis of chlorogenic acids, and can be determined by the skilled person by routine methods, e.g. using the methods disclosed in the examples herein.

In another embodiment the hydrolysis of chlorogenic acid is performed by the use of an enzyme capable of hydrolysing chlorogenic acids. A suitable enzyme is e.g. an esterase e.g. a chlorogenate esterase derived from Aspergillus japonicus. (Commercially available from Kikkoman, Japan), Tannase from Aspergillus oryzae (EC 3.1.1.20) (commercially available from Kikkoman, Japan); Palatase 20000L (EC 3.1.1.3) (commercially available from Novozymes A/S, Denmark). The enzymatic hydrolysis may be performed by conventional methods for enzymatic reactions, e.g. by dissolving or suspending the enzyme in the coffee extract under conditions suitable for the required enzyme activity. The enzyme may be inactivated, e.g. by heating, after the hydrolysis has taken place. The enzyme may also be immobilised, e.g. on a membrane or on an inert carrier, and the coffee extract to be treated may be circulated over the membrane or through the carrier until the desired degree of hydrolysis has been achieved.

The amount of enzyme and conditions to be used should be suitable to achieve the desired hydrolysis of chlorogenic acids, and can be determined by the skilled person by routine methods, e.g. using the methods disclosed in the examples herein to determine the hydrolysis of chlorogenic acids.

The invention also relates to a method of producing a food or beverage product wherein a coffee extract of the invention is used as an ingredient of said food or beverage product. The extract is used separately from the extracted coffee beans, i.e. undissolved material from the beans is substantially removed by separation as described herein and is not used in the production of the food or beverage product. The food or beverage product may be any food or beverage product known in the art. In a preferred embodiment the food or beverage product is a coffee product, e.g. a soluble coffee product or a ready-to-drink coffee product. A soluble coffee product may be produced by concentrating and drying the extract of the invention. Before drying, the extract may be mixed with coffee extract that has not been treated to hydrolyse chlorogenic acids, e.g. extract of roasted coffee beans, green coffee beans, or both. Methods for producing a soluble coffee product from coffee extract are well known in the art. When the extract is used for the production of a coffee product, the beans to be extracted may have been subjected to stripping to remove volatile aromas before extraction, e.g. as described in EP-A-1078576. The volatile aromas may then be added back to the extract after the treatment to hydrolyse chlorogenic acids, e.g. after drying to produce an aromatised soluble coffee product. A soluble coffee product produced from a coffee extract of the invention may be sold as such, or may e.g. be mixed with a creamer and/or sweetener and sold to prepare a coffee beverage comprising creamer and/or sweetener, e.g. cappuccino or café latte.

When a coffee extract according to the invention is used as an ingredient of a food or beverage product it may be added at any appropriate step in the production process of said food or beverage product to achieve the desired effect. The extract may be added in any amount suitable to bring about the desired effect, e.g. antioxidant effect. A food or beverage product produced by the method of the invention may e.g. be a coffee based beverage, a tea based beverage, a soft drink, a dairy product, a confectionery product, or a nutritional supplement.

The present invention also relates to the use of a coffee extract of the invention as an antioxidant, e.g. as an ingredient in a product, e.g. a food or beverage product, wherein antioxidant properties are desired, e.g. to prevent oxidation of components of the product during storage. Antioxidants are commonly used in a number of products and the coffee extract of the invention may be used in a similar way as conventional antioxidants.

The coffee extract of the invention may also be used to enhance antioxidant capacity in vivo in a human or animal, e.g. by inducing detoxifying enzymes such as gluthathione-S-transferase (GST) and by increasing the Nrf2-mediated gene expression pathway. Increased Nrf2 activity associated genes have been reported to enhance detoxification and to stimulate the endogenous defense against oxidative stress. These effects may e.g. be achieved by oral administration of the coffee extract or by topical application to the skin of a human or an animal.

The coffee extract of the invention may be used to decrease inflammation, e.g. by inhibiting the increase in prostaglandin E2 level by pro-inflammatory agents (e.g. interleukin 1b, lipopolysaccharides (LPS).

Many health problems and disorders are related to oxidative stress and inflammation. The coffee extract of the invention may be used to treat or prevent such problems or disorders. Relevant problems and disorders are e.g. skin disorders, e.g. photodamage caused by UV-radiation, atopic dermatitis, eczema, scaling, itching, allergic symptoms; brain disorders; inflammation; obesity; and cancer, e.g. skin cancer and lung cancer.

The coffee extract of the invention may further be used as an anti-diabetic agent, e.g. by reducing blood glucose levels, and/or increasing blood levels of leptin, insulin and/or c-peptide; as a bone remodelling agent, e.g. by increasing bone mineral density, e.g. by increasing serum levels of estrogen and/or progesterone and/or alkaline phosphatase activity; as anti-metastatic agents, e.g. with anti-angiogenic effect.

The coffee extract according to the invention may be used for the preparation of a formulation to treat or prevent skin disorders, diabetes, allergies, brain disorders, inflammation, obesity and/or cancer. The formulation may be in any suitable form, e.g. for oral administration or topical administration to the skin, e.g. in the form of a food or beverage product, a nutritional supplement, a tablet, a lotion, or a cosmetic product. In a preferred embodiment the formulation is a medicament.

### EXAMPLES

### Example 1

Treatment of NESCAFE PROTECT® with Lactobacillus johnsonii Fresh Cells

Cells of L. johnsonii (CNCM I-1225) were grown (7.0 E08 cfu/ml) and centrifuged (5000 g, 10 min), the pellets were resuspended in phosphate buffer (50 mM, pH 7.0) at a concentration of 0.61 g/ml. 30 mg/ml of NESCAFE PROTECT® (a dried co-extract of green and roasted coffee beans) was added and the mixture was incubated at 37° C. Samples were withdrawn at different reaction times, centrifuged (3000 g, 5 min) and filtered through 0.45 μm pore size syringe filters (Millipore SLHA 025 BS) and analysed by HPLC.

A reaction control was run in parallel under the same reaction conditions but without bacteria.

Treatment of NESCAFE PROTECT® with Lactobacillus johnsonii Extract (Lysed Cells)

Cells of L. johnsonii (CNCM I-1225) were grown (7.0 E08 cfu/ml) and centrifuged (5000 g, 10 min), the pellets were resuspended in phosphate buffer (50 mM, pH 7.0) at a concentration of 0.61 g/ml. The cells were then lysed using the glass-beads method. 600 μl of cells preparation were put in screw-cap tubes and 600 μl of glass-beads were added at 0° C. The tubes were then put into a Mini-Beadbeater for 1 min of intense shaking, cooled in ice, and put another 1 min in the Mini-Beadbeater. The crude cell extract was then added to 900 μl of a solution of NESCAFE PROTECT® (30 mg/ml, phosphate buffer pH 7.0) and the mixture was incubated at 37° C. Samples were withdrawn at different reaction times, centrifuged (3000 g, 5 min), filtered through 0.45 μm pore size syringe filters (Millipore SLHA 025 BS) and analysed by HPLC.

Treatment of NESCAFE PROTECT® with Spray-Dried Preparation of Lactobacillus johnsonii

30 mg of NESCAFE PROTECT® were dissolved in 1 ml phosphate buffer (50 mM, pH 7.0) or in 1 ml water. To this solution, 10 mg of a spray-dried preparation of Lactobacillus johnsonii (CNCM I-1225) (3.3 E9 cfu/g) were added. The mixture was then incubated at 37° C. and samples were withdrawn at different reaction times. After centrifugation (3000 g, 5 min) and filtration (0.45 μm pore size syringe filters, Millipore SLHA 025 BS) the samples were analysed by HPLC.

Treatment of Green Coffee Extract with a Spray-Dried Preparation of Lactobacillus johnsonii (CNCM I-1225)

30 mg of a dried green coffee extract was dissolved in 1 ml phosphate buffer (50 mM, pH 7.0) or in 1 ml water. To this solution, 10 mg of a spray-dried preparation of Lactobacillus johnsonii (3.3 E9 cfu/g) was added. The mixture was then incubated at 37° C. and samples were withdrawn at different reaction times. After centrifugation (3000 g, 5 min) and filtration (0.45 μm pore size syringe filters, Millipore SLHA 025 BS) the samples were analysed by HPLC.

Treatment of NESCAFE® with a Concentrated Preparation of Lactobacillus johnsonii (CNCM I-1225)

400 mg of NESCAFE SPECIAL FILTRE® (a dried extract of roasted coffee beans) were dissolved in 1 ml of boiling water and the solution was cooled to 37° C. at room temperature. To 250 μl of this coffee solution, different amounts of concentrated preparation of Lactobacillus johnsonii (50 μl, 100 μl, 350 μl, 750 μl) were added and the volume was adjusted to 1 ml with water. The mixtures were than incubated at 37° C. for 2 h and 4 h. After centrifugation (3000 g, 5 min) and filtration the samples were analysed by HPLC.

### HPLC Analysis

Coffee samples were diluted to 1% w/w and analyzed by RP-HPLC on a CC 250/4 Nucleosil 100-5-C18 column (Macherey-Nagel). The eluent system was Millipore water, 0.1% TFA and CH₃CN at a flow rate of 1 mL/min. The method allowed the simultaneous determination of caffeoyl quinic acids (CQA), feruloyl quinic acids (FQA), di-caffeoyl quinic acids (diCQA), feruloyl quinic acid-lactones, caffeic acid (CA) and ferulic acid (FA) (absorbance at 325 nm) using external standard calibration curves. Results were expressed relative to the reference at time 0 (t0) or to the reference at the same time without bacteria.

### Antioxidant Responsive Element (ARE) Luciferase Assay

The pGL-8×ARE which contains eight copies of the ARE present in rat glutathione-S-transferase A2 (GSTA2) along with the pcDNA3.1 plasmid containing the neomycin selectable marker was stably transfected into human MCF7 cells (Wang et al., Cancer Res. 66, 10983-10994, 2006). ARE (antioxidant-responsive element) is the binding site of the transcription factor Nrf2 which regulates the genes involved in detoxification and endogenous defense against oxidative stress. The plasmid pGL-8×ARE contains a luciferase gene downstream of the eight Nrf2 binding sites that allows monitoring Nrf2 activity.

For treatment with coffee, the AREc 32 cells were seeded in 96-well microtiter plates in DMEM growth medium. After treatment for 24 h with the different coffees, firefly luciferase activity was determined.

### Protein Expression

Primary hepatocytes were obtained by perfusion of the liver of Sprague-Dawley rats with a collagenase solution (Sidhu et al., Arch. Biochem. Biophys. 301, 103-113, 1993). Cell viability, estimated by Trypan Blue exclusion test, was found to range between 90-95%. The cells were seeded at a density of 1.5×10⁵ cells/cm² on 60 mm plastic tissue culture dishes in 3 ml of William's medium supplemented with 2 mM L-glutamine, 10 mM Hepes pH 7.4, ITS+, 15000U Penicillin/Streptomycin, 100 nM Dexamethasone and 5% Fetal bovine serum (Hi-clone). Hepatocytes were allowed to attach for two hours and then washed with EBSS to remove debris and unattached cells. Fresh serum-free medium containing 25 nM of dexamethasone was added and an overlay of matrigel (233 g/ml) was then applied. Fresh matrigel was added to the cultures every two days following medium change. To study the effect of coffee on detoxifying enzymes and antioxidant protein expression, the test material was added to the culture media 24 hours after cell seeding for a period of 48 hours before protein extraction and western blot analysis (Cavin et al., Food Chem Tox. 46, 1239-48, 2008).

### Prostaglandin E2 Formation Assay

Human colon HT-29 cells were treated with the different coffees for 15 h followed by a co-incubation of 6 h together with a pro-inflammatory agent TNF-α (10 ng/ml). Analysis of the PGE2 production in HT-29 cells was determined using a competitive enzyme immunoassay (EIA) (Cavin et al., BBRC 327, 742-49, 2005).

### Results

### Hydrolysis of Chlorogenic Acids to Generate Phenolic Acids

Experiment 1: Treatment of NESCAFE PROTECT® with L. johnsonii fresh cells with varying reaction times and amount of cell preparation. Results are shown in Table 1.

TABLE 1
Results of experiment 1.

| Time (h) | 6 | 24 | 6 | 24 | 6 | 24 |
|---|---|---|---|---|---|---|
| Cell preparation (uL) | 750 | 750 | 350 | 350 | 100 | 100 |

Concentration in % relative to
untreated reference at t = 0

| | | | | | | |
|---|---|---|---|---|---|---|
| CQA | 3 | 0 | 14 | 3 | 39 | 15 |
| FQA | 8 | 0 | 17 | 4 | 42 | 17 |
| diCQA | 0 | 0 | 0 | 0 | 23 | 2 |
| CA | 13215 | 13238 | 14282 | 15981 | 10111 | 13661 |
| FA | 7776 | 8845 | 7234 | 8861 | 4594 | 6691 |

Mass balance (mmol/g dry matter)

| | | | | | | |
|---|---|---|---|---|---|---|
| Chlorogenic acids | 0.20 | 0.21 | 0.18 | 0.20 | 0.13 | 0.17 |
| consumed | | | | | | |
| CA and FA | 0.20 | 0.20 | 0.21 | 0.24 | 0.14 | 0.20 |
| formed | | | | | | |

Experiment 2: Treatment of NESCAFE PROTECT® with L. johnsonii extract (lysed cells) with varying reaction times and amount of cell preparation. Results are shown in Table 2.

TABLE 2
Results of experiment 2.

| Time (h) | 2 | 2 | 6 | 6 |
|---|---|---|---|---|
| Cell preparation (uL) | 350.0 | 100.0 | 350.0 | 100.0 |

Concentration in % relative to
untreated reference at t = 0

| | | | | |
|---|---|---|---|---|
| CQA | 10 | 32 | 6 | 14 |
| FQA | 15 | 36 | 11 | 18 |
| diCQA | 1 | 8 | 1 | 1 |
| CA | 13901 | 10729 | 16300 | 12581 |
| FA | 7771 | 5581 | 9720 | 6960 |

Experiment 3: Treatment of NESCAFE PROTECT® with spray-dried preparation of Lactobacillus johnsonii. Results are shown in Table 3.

TABLE 3
Results of experiment 3.

| Time (h) | 2 | 6 | 24 |
|---|---|---|---|
| CQA | 73 | 67 | 32 |
| FQA | 82 | 60 | 34 |
| CA | 3598 | 6140 | 7879 |
| FA | 1109 | 2183 | 3686 |

CQA, FQA, CA and FA are given as % relative to untreated control at t = 0.

Experiment 4: Treatment of green coffee extract with a spray-dried preparation of Lactobacillus johnsonii. Results are shown in table 4.

TABLE 4
Results of experiment 4.

| Time (h) | 4 | 6 | 16 | 24 |
|---|---|---|---|---|
| CQA | 77 | 69 | 58 | 50 |
| FQA | 79 | 71 | 48 | 52 |
| diCQA | 67 | 53 | 32 | 20 |
| CA | 2673 | 3762 | 5182 | 6145 |
| FA | 961 | 1429 | 1963 | 2432 |

CQA, FQA, CA and FA are given as % relative to untreated control at t = 0.

Experiment 5: Treatment of NESCAFE® with a concentrated preparation of Lactobacillus johnsonii. Results are shown in table 5.

TABLE 5
Results of experiment 5.

| | Cell amount | | | | | | | |
|---|---|---|---|---|---|---|---|---|
| | 50 μL/ | 100 μL/ | 350 μL/ | 750 μL/ | 50 μL/ | 100 μL/ | 350 μL/ | 750 μL/ |
| | 1 mL | 1 mL | 1 mL | 1 mL | 1 mL | 1 mL | 1 mL | 1 mL |
| Time (h) | 2 | 2 | 2 | 2 | 4 | 4 | 4 | 4 |
| CQA | 92 | 80 | 46 | 25 | 76 | 60 | 33 | 17 |
| FQA | 90 | 82 | 61 | 41 | 89 | 74 | 53 | 37 |
| diCQA | 86 | 68 | 23 | 6 | 75 | 56 | 15 | 6 |
| CA | 1737 | 2885 | 5752 | 7292 | 1763 | 2803 | 4491 | 5514 |
| FA | 1509 | 2468 | 5327 | 7586 | 786 | 1266 | 2408 | 3281 |

CQA, FQA, CA and FA are given as % relative to untreated control at t = 0.

Table 6 show the absolute concentration of a number of compounds in two different samples of extracts of green coffee beans that have not been treated to hydrolyse chlorogeninc acids (control samples).

TABLE 6
Composition of untreated green coffee extracts (control samples).

| | A | B |
|---|---|---|
| 3-caffeoyl quinic acid | 13.88 | 15.57 |
| 4-caffeoyl quinic acid | 17.58 | 20.08 |
| 5-caffeoyl quinic acid | 81.16 | 85.45 |
| Sum CQA | 112.62 | 121.10 |
| 3-feruloyl quinic acid | 0.00 | 0.00 |
| 4-feruloyl quinic acid | 3.29 | 4.41 |
| 5-feruloyl quinic acid | 17.70 | 19.77 |
| Sum FQA | 20.99 | 24.18 |
| CA | 0.39 | 0.47 |
| FA | 0.25 | 0.23 |
| 3-caffeoyl quinic acid lactone | 0.00 | 0.00 |
| 4-caffeoyl quinic acid lactone | 0.00 | 0.00 |
| Sum Lactones | 0.00 | 0.00 |
| 3,4-dicaffeoyl quinic acid | 6.80 | 4.34 |
| 3,5-dicaffeoyl quinic acid | 5.53 | 8.35 |
| 4,5-dicaffeoyl quinic acid | 0.12 | 8.85 |
| sum dicaffeoyl quinic acid | 12.45 | 21.55 |

Concentration is given in milligram per gram of dry matter.

### Protein Expression

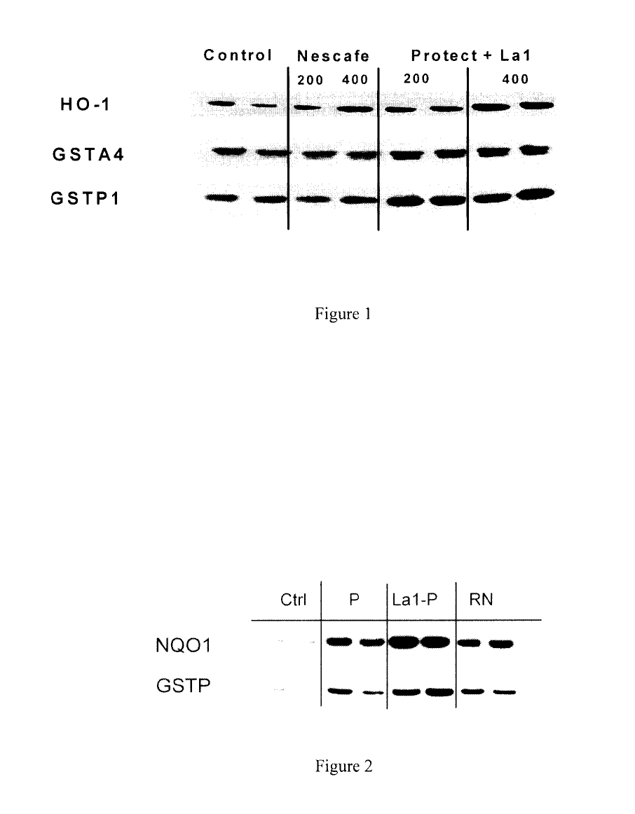

In rat primary hepatocytes, NESCAFE RED CUP® (extract of roasted coffee beans) at 200 ug/ml produced after 48 h of treatment no increase in the protein expression of GST subunits (GSTA4, GSTP1) and Heme-Oxygenase-1 (HO-1) and weak inductions of GSTP1 and HO-1 expressions at 400 ug/ml by western blot. In contrast, a stronger induction of the different protein expressions was observed with NESCAFE PROTECT® treated with L. johnsonii at both 200 ug/ml and 400 ug/ml on GSTA4, GSTP1 and HO-1). Results are shown as Western Blot gels in FIG. 1.

Data obtained in the liver of male rats fed in their diet for 2 weeks with 5% of NESCAFE RED CUP® versus NESCAFE PROTECT® and NESCAFE PROTECT® treated with L. johnsonii confirmed the effects observed in rat primary hepatocytes. Strongest induction of detoxifying enzyme expression (GSTP1; NQO1) was found with the NESCAFE PROTECT® treated with L. johnsonii as compared to untreated NESCAFE PROTECT® (GSTP1; NQO1) and untreated NESCAFE RED CUP® (GSTP1; NQO1). Results are shown as Western Blot gels in FIG. 2.

### Antioxidant Responsive Element (ARE) Luciferase Assay

Human breast cancer cells (AREc32) stably transfected with several copies of the rat GSTA2-ARE reporter construct was used to demonstrate the activation of Nrf2-ARE pathway by coffee. Green coffee extract not treated to hydrolyse chlorogenic acids, and different green coffee extract treated with L. johnsonii for 24 h produced a dose-dependent increase in Nrf2-luciferase reporter activity (see table 7).

TABLE 7
Induction of Nrf2 activity by coffee
(firefly luciferase activity, AU).

| Coffee | Green coffee | Green coffee | Green coffee | Green coffee |
|---|---|---|---|---|
| extract | extract | extract 1 | extract 2 | extract 3 |
| ug/ml | untreated | treated | treated | treated |
| 0 | 0 | 0 | 0 | 0 |
| 200 | 0.3 +/− 0.1 | 15.2 +/− 1.5 | 22.1 +/− 1.6 | 22.6 +/− 2.4 |
| 300 | 0.8 +/− 0.1 | 29.0 +/− 3.0 | 40.5 +/− 1.6 | 32.1 +/− 6.0 |
| 400 | 1.0 +/− 0.1 | 40.4 +/− 6.6 | 59.3 +/− 1.9 | 47.3 +/− 3.4 |
| 600 | 2.0 +/− 0.2 | 77.7 +/− 10.5 | 90.4 +/− 0.7 | 80.4 +/− 8.2 |
| 800 | 2.8 +/− 0.1 | 77.3 +/− 4.9 | 80.7 +/− 7.3 | 96.2 +/− 4.1 |

### Prostaglandin E2 Formation Assay

Potential anti-inflammatory effect of green coffee extract treated with L. johnsonii was assessed in human colon HT-29 cells. Following treatment with a pro-inflammatory agent TNF-α, prostaglandin E2 (PGE₂) level is induced in colon cells. In this study, cells were pre-treated for 24 h with different coffee extracts (green coffee extract not treated to hydrolyse chlorogenic acids, and different green coffee extract treated with L. johnsonii for 24 h). TNF-α (10 ng/ml) was added in the last 6 h of the experiment. Data (see table 8) showed a clear dose-dependent decrease by coffees of PGE₂ formation as compared to control cells treated with TNF-α

TABLE 8
Decrease of PGE₂ formation as a result of coffee extract treatment
as compared to control cells treated with TNF-α (AU).

| Coffee | Green coffee | Green coffee | Green coffee |
|---|---|---|---|
| extract | extract | extract 1 | extract 2 |
| ug/ml | untreated | treated | treated |
| 0 | 100 +/− 9 | 100 +/− 9 | 100 +/− 9 |
| 50 | 110 +/− 10 | 18 +/− 2 | 43 +/− 5 |
| 100 | 85 +/− 9 | 6 +/− 0.9 | 10 +/− 1.1 |
| 200 | 80 +/− 8 | 1 +/− 0.1 | 1 +/− 0.2 |

### Example 2

### Coffee Samples

Green coffee extract from 100% Robusta green beans NESCAFE PROTECT®, a dried co-extract of green and roasted coffee beans

Enzymes and cells

| Microorganisms | Culture medium |
|---|---|
| Lactobacillus johnsonii (CNCM I-1225) | MRS |
| Bifidobacterium lactis BB12 (CNCM I-3446) | MRS + cysteine |
| Bifidobacterium longum BB536 (ATCC BAA-999) | MRS + cysteine |

Chlorogenate esterase (24 U/g), derived from Aspergillus japonicus (Kikkoman, Japan).
Tannase from Aspergillus oryzae (Kikkoman, Japan)

### Preparation of Bacteria Cells

Tested strains were harvested (centrifugation at 5000 g for 10 min) after having well reached stationary phase, corresponding to 16 hours of incubation in culture medium at 37° C. in anaerobic atmosphere without agitation. For a first activation of the strains, frozen stock cultures were inoculated in fresh media and grown overnight. This pre-culture was used to inoculate the culture.

Treatment of Coffee Extracts with Bacteria Cells

After culture of bacteria and centrifugation, the pellets were resuspended in phosphate buffer (pH 7.0) at a concentration of 0.61 g/ml. To 200 μl of this cells preparation, 800 μl of a coffee solution (3%) was added and the mixture was incubated at 37° C. for 4 h, 16 h and 24 h.

Incubation of Coffee Extracts with Chlorogenate Esterase

A solution of chlorogenate esterase (25 mg) in 200 μl phosphate buffer (pH 7.0) was added to 800 μl of a coffee solution (3%). The mixture was then incubated at 37° C. for 4 h, 16 h and 24 h. After reaction time, the enzymatic activity was stopped by heat treatment (3 min, 90° C.) and the mixture was filtered before analysis.

### ARE Luciferase Assay

As in example 1

### Results

Human breast cancer cells (AREc32) stably transfected with several copies of the rat GSTA2-ARE reporter construct was used to demonstrate the activation of the antioxidant Nrf2-ARE pathway by coffee. Green coffee extract not treated to hydrolyse chlorogenic acids (untreated), green coffee extract treated with Lactobacillus johnsonii (Lj) for 24 h, green coffee extract treated with Bifidobacterium lactis (Bl) for 24 h, and green coffee extract treated chlorogenate esterase (CE) for 4 h, all produced a dose-dependent increase in Nrf2-luciferase reporter activity (table 9).

TABLE 9
Nrf2-luciferase reporter activity of untreated
and treated coffee extracts (AU).

| | | Green coffee | Green coffee | Green coffee |
|---|---|---|---|---|
| Coffees | Green coffee | treated with | treated with | treated with |
| mg/ml | untreated | Lj | Bl | CE |
| 0 | 0 | 0 | 0 | 0 |
| 100 | 0.3 +/− 0.1 | 0.5 +/− 0.1 | 0.3 +/− 0.1 | 1.0 +/− 0.1 |
| 200 | 0.8 +/− 0.1 | 1.0 +/− 0.1 | 1.1 +/− 0.1 | 2.1 +/− 0.2 |
| 400 | 1.0 +/− 0.1 | 5.2 +/− 0.4 | 3.1 +/− 0.3 | 8.2 +/− 1.4 |
| 600 | 2.0 +/− 0.2 | 11.1 +/− 0.5 | 7.2 +/− 1 | 11.3 +/− 1.4 |

Green coffee extracts were treated with different microrganisms and chlorogenate esterase to hydrolyse chlorogenic acids. Results are shown in table 10.

TABLE 10
Composition of green coffee extracts as a result of treatments.

| | Lactobacillus | | | Bifidobacterium | | | Chlorogenate | | |
|---|---|---|---|---|---|---|---|---|---|
| | Johnsonii | | | lactis | | | esterase | | |
| time (h) | 4 | 16 | 24 | 4 | 16 | 24 | 4 | 16 | 24 |
| CQA | 42 | 15 | 12 | 96 | 79 | 74 | 3 | 2 | 2 |
| FQA | 49 | 15 | 15 | 33 | 12 | 6 | 73 | 24 | 23 |
| diCQA | 35 | 6 | 3 | 85 | 70 | 64 | 0 | 0 | 0 |
| CA | 12505 | 17148 | 18107 | 1521 | 4182 | 5283 | 18917 | 17275 | 18318 |
| FA | 4607 | 7192 | 7597 | 5020 | 6970 | 7105 | 2620 | 5685 | 6293 |

CQA, FQA, CA and FA are given as % relative to untreated control at t = 0.

NESCAFE PROTECT® was treated with different microrganisms and chlorogenate esterase to hydrolyse chlorogenic acids. Results are shown in table 11.

TABLE 11
Composition of NESCAFE PROTECT ® as a result of treatments.

| | Lactobacillus | | | Bifidobacterium | | | Chlorogenate | | |
|---|---|---|---|---|---|---|---|---|---|
| | Johnsonii | | | lactis | | | esterase | | |
| time (h) | 4 | 16 | 24 | 4 | 16 | 24 | 4 | 16 | 24 |
| CQA | 33 | 13 | 13 | 97 | 81 | 80 | 5 | 3 | 3 |
| FQA | 42 | 21 | 20 | 54 | 23 | 20 | 54 | 15 | 14 |
| diCQA | 18 | 2 | 2 | 91 | 72 | 67 | 0 | 0 | 1 |
| CA | 7942 | 9429 | 9933 | 968 | 2258 | 2840 | 10879 | 10198 | 10750 |
| FA | 4051 | 5226 | 5504 | 3065 | 4518 | 5144 | 2794 | 5140 | 5518 |

CQA, FQA, CA and FA are given as % relative to untreated control at t = 0.

1. A method of producing a coffee extract, comprising the steps:
a) extracting coffee beans with water to produce a coffee extract; and
b) treating the coffee extract to hydrolyse chlorogenic acids and generate phenolic acids.

2. The method of claim 1 wherein the coffee beans are whole coffee beans.

3. The method of claim 1 wherein the coffee beans are green coffee beans.

4. The method of claim 3 wherein the green coffee beans are co-extracted with roasted coffee beans in step a).

5. The method of claim 1 wherein the coffee extract is separated from the extracted coffee beans at a time selected from the group consisting of before, during and after the treatment of step b).

6. The method of claim 5 wherein the coffee extract is kept separate from the extracted coffee beans after the treatment in step b).

7. The method of claim 3 wherein a green coffee extract obtained in step a) is mixed with an extract of roasted coffee beans, the mixture being treated in step b).

8. The method of claim 1 wherein the hydrolysis of chlorogenic acid in step b) is performed by fermentation with a microorganism capable of hydrolysing chlorogenic acid to generate phenolic acids.

9. A method of producing a food or beverage product wherein a coffee extract produced by the steps:
extracting coffee beans with water to produce a coffee extract; and
treating the coffee extract to hydrolyse chlorogenic acids and generate phenolic acids; and
using the extract as an ingredient to produce the food or beverage product.

10. A method comprising using as an antioxidant a coffee extract comprising an amount of caffeic acid that is at least 1 milligram per gram of dry matter and/or the amount of ferulic acid is at least 0.5 milligram per gram of dry matter.

11. A method for producing a medicament comprising the step of using a coffee extract comprising an amount of caffeic acid that is at least 1 milligram per gram of dry matter and/or the amount of ferulic acid is at least 0.5 milligram per gram of dry matter.

12. A method for treating or preventing a condition selected from the group consisting of skin disorders, diabetes, brain disorders, inflammation, obesity and cancer comprising administering to an individual in need of same a composition comprising a coffee extract comprising an amount of caffeic acid that is at least 1 milligram per gram of dry matter and/or the amount of ferulic acid is at least 0.5 milligram per gram of dry matter 5.

13. A method for promoting bone remodelling comprising administering to an individual in need of same a composition comprising a coffee extract comprising an amount of caffeic acid that is at least 1 milligram per gram of dry matter and/or the amount of ferulic acid is at least 0.5 milligram per gram of dry matter.

14. Method according to claim 10 wherein the ratio of the amount of caffeoyl quinic acids and diesters to the amount of caffeic acid in the coffee extract is less than 100 (weight/weight).

15. Method according to claim 10 wherein the ratio of the amount of feruloyl quinic acids and diesters to the amount of ferulic acid in the coffee extract is less than 30 (weight/weight).

16. Method according to claim 11 wherein the ratio of the amount of caffeoyl quinic acids and diesters to the amount of caffeic acid in the coffee extract is less than 100 (weight/weight).

17. Method according to claim 11 wherein the ratio of the amount of feruloyl quinic acids and diesters to the amount of ferulic acid in the coffee extract is less than 30 (weight/weight).

18. Method according to claim 12 wherein the ratio of the amount of caffeoyl quinic acids and diesters to the amount of caffeic acid in the coffee extract is less than 100 (weight/weight).

19. Method according to claim 12 wherein the ratio of the amount of feruloyl quinic acids and diesters to the amount of ferulic acid in the coffee extract is less than 30 (weight/weight).

20. Method according to claim 13 wherein the ratio of the amount of caffeoyl quinic acids and diesters to the amount of caffeic acid in the coffee extract is less than 100 (weight/weight).

21. Method according to claim 13 wherein the ratio of the amount of feruloyl quinic acids and diesters to the amount of ferulic acid in the coffee extract is less than 30 (weight/weight).
